(19) United States
(12) Patent Application Publication
(10) Pub. No.: US 2010/0099198 A1
(43) Pub. Date: Apr. 22, 2010

(54) APPARATUS AND SYSTEM FOR PATTERN RECOGNITION SENSING FOR BIOMOLECULES

(75) Inventors: Qitao Zhao, Arlington, TX (US); Dilani A. Jayawardhana, Arlington, TX (US); Xiyun Guan, Arlington, TX (US)

Correspondence Address: CHALKER FLORES, LLP, 2711 LBJ FRWY, Suite 1036, DALLAS, TX, 75234, US

(73) Assignee: Board of Regents, The University of Texas System, Austin, TX (US)

(21) Appl. No.: 12/499,725

(22) Filed: Jul. 8, 2009

Related U.S. Application Data
(60) Provisional application No. 61/079,864, filed on Jul. 11, 2008.

(51) Int. Cl. G01N 27/416; C12M 1/00; H01R 43/00

(52) U.S. Cl. 436/149; 422/82.01; 435/287.1; 29/825

(57) ABSTRACT

The present invention is an array nanopore stochastic sensing system for detection of single biomolecules and oligonucleotides. The system comprises a multi-channel system with multiple genetically modified protein pores for detection of analytes using the pattern recognition mechanism. By monitoring current blockade patterns, identity of single biomolecules can be determined in complex mixtures.

### CROSS-REFERENCE TO RELATED APPLICATIONS

This application claims benefit of U.S. Provisional Application Ser. No. 61/079,864, filed Jul. 11, 2008, which is incorporated herein by reference in its entirety.

### STATEMENT OF FEDERALLY FUNDED RESEARCH

This invention was made with U.S. Government support under Contract No. W911NF-06-1-0240 awarded by the DARPA. The government has certain rights in this invention.

### TECHNICAL FIELD OF THE INVENTION

The present invention relates in general to the field of bimolecular sensing, and more particularly, to the design and application of devices comprising an array of stochastic nanopore sensors based on the pattern recognition mechanism.

### BACKGROUND OF THE INVENTION

Without limiting the scope of the invention, its background is described in connection with the design and applications of sensing devices comprising of an array of nanopore stochastic sensors based on the pattern recognition mechanism for the detection of biomolecules.

U.S. Pat. No. 7,005,264 issued to Su and Berlin (2006) describes a method and apparatus for sequencing and/or identifying nucleic acids. According to the '264 patent nucleic acids containing labeled nucleotides may be synthesized and passed through nanopores. Detectors operably coupled to the nanopores may detect the labeled nucleotides. By determining the time intervals at which labeled nucleotides are detected, distance maps for each type of labeled nucleotide may be compiled. The distance maps in turn may be used to sequence and/or identify the nucleic acid. In different embodiments of the invention, luminescent nucleotides or nanoparticles may be detected using photodetectors or electrical detectors. Apparatus and sub-devices of use for nucleic acid sequencing and/or identification are also disclosed.

United States Patent Application No. 20070178507 (Wu et al., 2007) discloses a molecular analysis device comprising a molecule sensor and a nanopore that passes through, partially through, or substantially near the molecule sensor. The molecule sensor may comprise a single electron transistor including a first terminal, a second terminal, and a nanogap or at least one quantum dot positioned between the first terminal and the second terminal. The molecular sensor may also comprise a nanowire that operably couples a first and a second terminal. A nitrogenous material that may be disposed on at least part of the molecule sensor is configured for a chemical interaction with an identifiable configuration of a molecule. The molecule sensor develops an electronic effect responsive to a molecule or responsive to a chemical interaction.

### SUMMARY OF THE INVENTION

In one embodiment the present invention describes a single molecule chemical sensing apparatus comprising: at least two cis chambers; at least one trans chamber; two or more boundary layers on a Teflon septum separating the cis and trans chambers; at least one pore selected from a porous synthetic membrane, or a wild type or genetically modified bacterial transmembrane protein attached to the boundary layer; one or more holes for addition of one or more solutions, at least three electrodes to the one or more chambers; at least two or more switches for monitoring an ionic current output; and a metal box for enclosing the entire apparatus.

In one aspect, the present invention the septum has a hole having a diameter ranging from 100-200 μm. In another aspect, a conducting electrolyte and an analyte are present in at least one of the chambers. In another aspect, the boundary layer comprises a lipid bi-layer or is a natural or synthetic membrane. In yet another aspect, the ionic current output is measured from at least two chambers sequentially or simultaneously.

One aspect of the invention describes a wild type or modified bacterial transmembrane protein comprising at least one or more of α-hemolysin, streptolysin, listeriolysin, leukocidin, binary toxins, aerolysin, cholesterol-dependent cytolysins, pneumolysins, or combinations thereof. Another aspect describes the conducting solution comprising a buffer, ionic salts, organic ion conducting solutions or combinations thereof. One aspect of the invention describes a pattern recognition mechanism sensing, wherein analytes are detected by a priori knowledge, statistical patterns, multidimensional spatial analysis, or combinations thereof. One aspect describes the analytes that can be detected. The analytes are unknown, known, or a combination. In yet another aspect the types of analytes are described. They can be biomolecules, oligonucleotides, environmental contaminants, bioterrorist agents, or combinations thereof. Biomolecular analytes comprise one or more proteins, peptides, fusion proteins, cells, monoclonal antibodies, polyclonal antibodies, receptors, growth-factors, hormones, or combinations thereof. One aspect describes bioterrorist agent, comprising one or more toxins, liquid explosives, toxins including neurotoxins and anthrax, cholinergic agents, TNT or combinations thereof. In yet another aspect analytes can be environmental contaminant, comprising one or more, heavy metals, cations, toxic chemicals, polymeric compounds, or combinations thereof. Analytes can also be oligonucleotides, comprising one or more, ssDNA, RNA, double stranded DNA, polynucleotides, or combinations thereof.

One aspect of the present invention describes a procedure of making one or more genetically modified bacterial transmembrane protein toxin and made by cassette mutagenesis comprising the steps of: cleaving a bacterial plasmid by a restriction enzyme to form an excised internal fragment and a plasmid with sticky ends; replacing the excised internal fragment by an oligonucleotide containing a sense and an antisense fragment; and inserting by ligation the sticky ends of the bacterial plasmid and the oligonucleotide to form a genetically modified bacterial transmembrane protein toxin. In another aspect the restriction enzyme comprises, one or more enzymes selected from EcoRI, EcoRII, BamHI, HindIII, TaqI, NotI, HinfI, Sau3A, PovII, SmaI, HaeIII, AluI, HpaI, SacII, EcoRV, KpnI, PsfI, SacI, SalI, ScaI, SphI, StuI, XbaI, and combinations thereof. Yet another aspect describes the method for making one or more genetically modified bacterial transmembrane α-hemolysin by cassette mutagenesis comprising the steps of: cleaving a bacterial plasmid pT7-αHL-RL2 position by restriction enzymes SacII and HpaI to form an excised fragment and a plasmid with sticky ends; replacing the excised internal fragment with a duplex DNA formed comprising a sense and antisense fragments; and inserting by ligation the sticky ends of the bacterial plasmid and the duplex DNA to form a genetically modified transmembrane α-hemolysin.

Another embodiment of the present invention is a method of detecting the presence of one or more single-molecules utilizing a multi-channel chemical sensing apparatus comprising the steps of: dissolving the one or more analytes in the sample in water or a buffer solution comprising an ionic salt to form a solution; placing the solution in a trans compartment of a multi-channel sensor; contacting the solution with at least two or more pore assemblies comprising a wild type or genetically modified bacterial transmembrane protein toxin; applying an electrical potential to the multi-channel sensor; determining an ionic current across the electrical potential; measuring one or more transient blockades in the ionic current; and comparing the transient blockades in the ionic current to one or more known transient current blockades to determine the identity of the one or more analytes.

In yet another embodiment the present invention described a method for fabricating a multi-channel chemical sensing apparatus for detecting single molecules, comprising the steps of: depositing at least two bilayers of a lipid molecule in an aperture of at least two or more Teflon septa; forming the bilayer at an air-water interface by hydrophobic apposition and the joining of the hydrocarbon chains of the individual monolayers; monitoring the bilayer formation using a function generator; and adding at least two or more pore selected from a wild type bacterial transmembrane protein or a modified bacterial transmembrane protein to at least two or more of the bilayers or utilizing porous synthetic membranes; adding the conducting electrolyte to the chambers; drilling one or more holes for adding one or more solutions; drilling one or more holes for placing at least three electrodes; attaching at least two switches; and enclosing the apparatus in a metal box.

### BRIEF DESCRIPTION OF THE DRAWINGS

For a more complete understanding of the features and advantages of the present invention, reference is now made to the detailed description of the invention along with the accompanying figures and in which:

FIG. 1 shows an α-hemolysin pore structure;

FIG. 2 is a schematic representation of the nanopore stochastic sensing mechanism;

FIG. 3 shows a two-channel nanopore sensor comprising three compartments: side-view of the two-channel device, with three compartments with two Teflon films with modified protein pores (3A); top view of the two-channel device comprising left (cis) and middle (trans) compartments—Channel 1 and right (cis) and middle (trans) compartment—Channel 2. Mixture to be analyzed is added to the trans compartment. The schematic also shows the three electrodes (3B); electrical connections and the switches associated with the nanopore device (3C); photograph of the nanopore device (3D); and pattern recognition sensing of two analytes 100 μM Diethylenetriaminepentamethylenephosphonic acid heptasodium salt (DTPMPA), or 1 μM Y-Y-Y-Y-Y-Y (Y6) (SEQ ID NO.: 1) peptide. The studies were performed at −40 mV in 1M NaCl and 10 mM Tris.HCl (pH 7.5);

FIG. 4 is an illustration of a pattern recognition stochastic sensor consisting of four protein nanopores: a). a sensing chamber, which has four cis compartments, labeled as 1, 2, 3, and 4, and one trans compartment 5, b). formation of a lipid bilayer along the 150 μm hole of the Teflon film, c). insertion of a single αHL pore into the lipid bilayer (4A); schematic configuration of the four proteins after their insertion into the lipid bilayers formed on the apertures of the Teflon films which separate the cis and trans compartments (4B); electrical connections and the switches associated with the nanopore device (4C);

FIG. 5 shows the formation of lipid bilayers and insertion of concurrent single channels: formation of four lipid bilayers on the apertures of the Teflon films (5A); insertion of four single channels into four lipid bilayers (5B); and the corresponding all-points histogram of 5B (5C). The four protein pores used were sensor 1: (M113F)₇(T145F)₇(K147N)₇; sensor 2: (M113E)₇; sensor 3: (M113R)₇(T145R)₇, and sensor 4: (WT)₇. The studies were performed at −40 mV (cis at ground) with 1 M NaCl and 10 mM Tris.HCl (pH 7.5) with the switches being turned on sequentially and/or then turned off sequentially;

FIG. 6 shows the electrical recordings showing the current blockages of various analytes in the four component pores of the pattern-recognition stochastic sensor. The four protein pores used were sensor 1: (M113F)₇(T145F)₇(K147N)₇; sensor 2: (M113E)₇; sensor 3: (M113R)₇(T145R)₇, and sensor 4: (WT)₇. The studies were performed at +40 mV or −40 mV (cis at ground) with 1 M NaCl and 10 mM Tris.HCl (pH 7.5);

FIG. 7 shows the pattern-recognition differentiation of a variety of molecules: dwell time plot (7A); and amplitude plot (7B). The four protein pores used were sensor 1: (M113F)₇(T145F)₇(K147N)₇ (i.e., (2FN)₇); sensor 2: (M113E)₇; sensor 3: (M113R)₇(T145R)₇ (i.e., (2R)₇), and sensor 4: (WT)₇. The studies were performed at +40 mV or −40 mV (cis at ground) with 1 M NaCl and 10 mM Tris.HCl (pH 7.5);

FIG. 8 shows the simultaneous detection of a mixture of two analytes. The two protein pores used were sensor 1: (M113R)₇(T145R)₇; and sensor 2: (M113F)₇(T145F)₇(K147N)₇. The studies were performed at −40 mV (cis at ground) under symmetrical buffer conditions with 1 M NaCl and 10 mM Tris.HCl (pH 7.5). 10 μM cyclo(P-G)₃ and/or 20 μM DTPMPA was added in the trans compartment of the chamber; and

FIG. 9 shows the identification of analytes in a double-channel consisting of two different single protein pores: amplitude histograms of DTPMPA in a single (M113R)₇(T145R)₇ pore (left) and cyclo(P-G)₃ in a single (M113F)₇(T145F)₇(K147N)₇ channel (right) (9A); amplitude histograms of DTPMPA (left) and cyclo(P-G)₃ (right) in a double-channel consisting of a single (M113R)₇(T145R)₇ pore and a single (M113F)₇(T145F)₇(K147N)₇ protein (9B); amplitude histograms of a mixture of DTPMPA and cyclo(P-G)₃ in a double-channel consisting of a single (M113R)₇(T145R)₇ pore and a single (M113F)₇(T145F)₇(K147N)₇ protein (9C). The experiments were performed at −40 mV (cis at ground) under symmetrical buffer conditions with 1 M NaCl and 10 mM Tris.HCl (pH 7.5). 10 μM cyclo(P-G)₃ and/or 20 μM DTPMPA was added in the trans compartment of the chamber.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

Nanopore stochastic sensing is a highly sensitive, rapid, and multifunctional sensing system that employs a biological protein pore embedded in a planar lipid bilayer or a fabricated nanoscale solid-state pore and a single-channel recording. Individual binding events are detected as current modulations. Genetically engineered versions of α-hemolysin (α-HL) have been used as stochastic sensing elements [1] for the identification and quantification of a wide variety of substances including the following: anions, organic molecules, explosives, enantiomers, proteins, DNA, and reactive molecules, divalent metal cations metal ions Zn(II), Co(II), and Cd(II), etc.[2-9]

The present invention employs pattern recognition mechanism for differentiation and detection of biomolecules and other compounds. This enables simultaneous detection of analytes in complex mixtures. The pattern-recognition nanopore sensor is a single sample compartment device controlled by a series of on/off switches.

Another feature of the present invention is the design of the pattern-recognition nanopore sensor array. Current technology is predominantly based on a single nanopore, including synthetic and biological pores. The present invention describes a nanopore array system, comprising of independent and parallel individual nanopores. The array design further enhances the capability of the pattern-recognition nanopore sensor to detect target compounds in complex mixtures and achieve simultaneous detection with reduced sample volumes.

The present invention also describes two pattern recognition nanopore array designs: i) a two-channel device comprising of three compartments with three electrodes, with two compartments having membranes containing the α-hemolysin protein pores, and ii) a four-channel device with five compartments with five electrodes, with four compartments having membranes containing the α-hemolysin protein pores.

The mammalian olfactory system can distinguish thousands of individual odors. High sensitivity and discrimination is achieved by an array of nonspecific cross-reactive receptors with different affinities for the analytes of interest [10]. In such a system, an odor is sensed by millions of sensory receptor neurons in the olfactory epithelium, and the resulting temporal response pattern from many receptor cells is then transmitted to the brain for processing and analysis. This biological sensing principle has been incorporated into a variety of chemical sensors, including electronic nose [11-12], in which an array of semiselective sensors coupled with a pattern-recognition algorithm are employed to identify and discriminate different compounds.

The present invention describes a single molecule chemical sensing system based on a pattern-recognition nanopore sensor array. Nanopore stochastic sensing is a highly-sensitive, rapid and multi-functional sensing system [13], that employs a biological protein pore embedded in a planar lipid bilayer or a fabricated nanoscale solid-state pore and single-channel recording. Individual binding events are detected as current modulations. Unlike other array sensors, such as piezoelectric [14], surface-acoustic wave [15], electrochemical [16], conducting polymer [17], and colorimetric variants [18], in which only one single parameter (i.e., signal intensity) is monitored, nanopore sensors can collect different types of information simultaneously from a single measurement, including event dwell time, amplitude, and voltage dependence. With an increase in the dimensionality of the sensing system, nanopore technology should provide superior resolution as a multi-analyte sensing method.

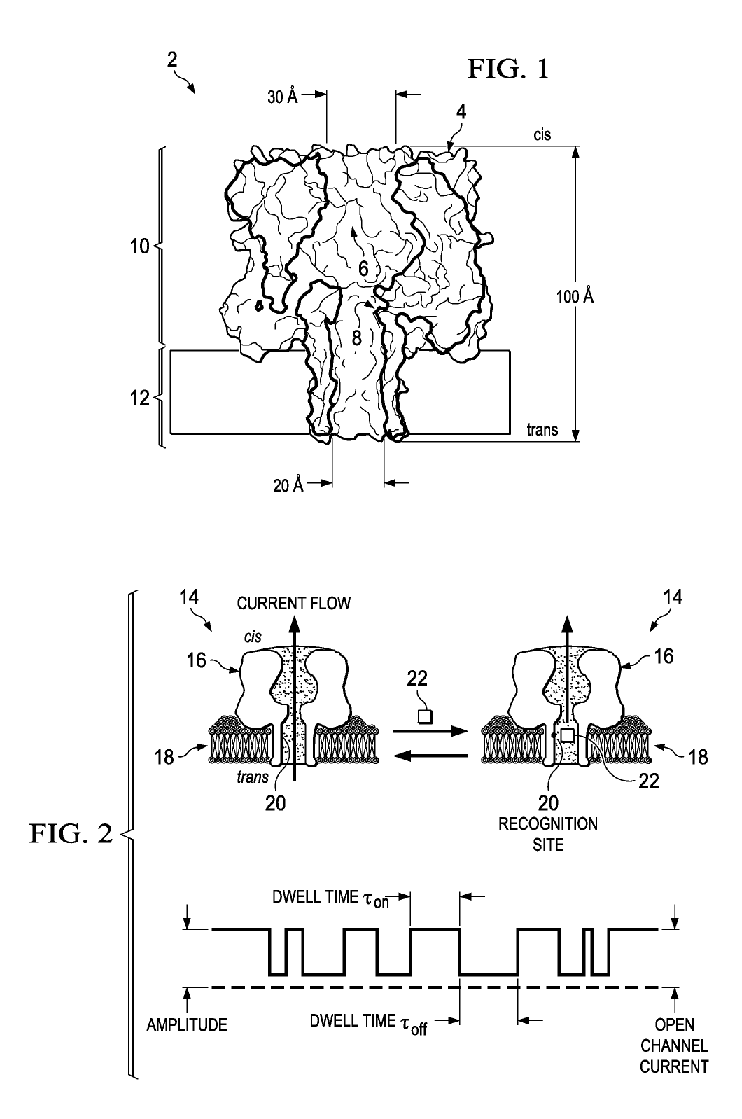

Nanopore stochastic sensing employs a biological protein pore embedded in a planar lipid bilayer or a fabricated nanoscale solid-state nanopore, coupled with single-channel recording [13, 19-21]. The most often used stochastic sensor element is a single transmembrane protein α-hemolysin (αHL) channel 2 as shown in FIG. 1. The wild-type αHL 4 forms a mushroom-shaped pore, which consists of seven identical subunits arranged around a central axis. The wild-type αHL 4 has a cap 10, a vestibule cavity 6, and a constriction 8. The opening of the channel on the cis side of the bilayer measures 29 Å in diameter and broadens into a cavity of ˜41 Å across. The cavity is connected to the trans-membrane domain, a 14-stranded β-barrel 12 with an average diameter of 20 Å (FIG. 1). Stochastic detection is achieved by monitoring the ionic current flowing through the single pore at an applied potential bias. Individual binding events are detected as transient blockades in the recorded current. This approach reveals both the concentration and the identity of an analyte. The former is obtained from the frequency of occurrence (1/τₒₙ) of the binding events and the latter by its characteristic current signature, typically the dwell time (τ_{off}) of the analyte coupled with the extent of current block (amplitude) it creates (FIG. 2). FIG. 2 shows the mechanism of stochastic sensing. A single transmembrane protein α-hemolysin (αHL) channel 14 comprises a wild-type or engineered αHL 16 and a bilayer 18. The recognition site and the analyte to be detected are depicted in 20 and 22 respectively. The direction of the flow of the ionic current is shown by the vertical arrow.

In this way, a wide variety of substances have been identified and quantified, including cations [9], anions [2], organic molecules [3], explosives [4], enantiomers [5], proteins [6], DNA [7, 22-25], and reactive molecules [8]. In stochastic sensing, since each analyte produces a characteristic signature, the sensor element itself need not be highly selective. Theoretically, this allows several analytes to be quantified concurrently using a single sensor element, as long as the sensor itself can provide enough resolution [9]. To further improve the resolution of the nanopore sensor for the differentiation of large molecules, particularly those that differ only slightly in composition, and even in the analysis of complex mixtures, in this work, a pattern-recognition approach was introduced into the nanopore technology.

Peptides Y-Y-Y-Y-Y-Y (SEQ ID NO.: 1), Y-P-F-F (SEQ ID NO.: 2), and HIV-1 TAT protein peptide (TATp) with a sequence of Y-G-R-K-K-R-R-Q-R-R—R (SEQ ID NO.: 3) were purchased from American Peptide Company, Inc. (Sunnyvale, Calif.). Organophosphate Diethylenetriaminepentamethylenephosphonic acid heptasodium salt (DTPMPA) and peptide cyclo(P-G)₃ were obtained from Sigma (St. Louis, Mo.). All these analytes were dissolved in HPLC-grade water (ChromAR, Mallinckrodt Baker. The stock solution of Y-Y-Y-Y-Y-Y (SEQ ID NO.: 1), was prepared at a concentration of 0.5 mM, and the stock solutions of all the other analytes were prepared at 1 mM each. All other reagents were purchased from Sigma.

Mutant αHL genes were constructed by site-directed mutagenesis (Mutagenex, Piscataway, N.J.) with a WT αHL gene in a T7 vector (pT7αHL), which has been described elsewhere [26]. Mutant αHL monomers were first synthesized by coupled in vitro transcription and translation (IVTT) using the E. coli T7 S30 Extract System for Circular DNA from Promega (Madison, Wis.). Subsequently, they were assembled into homoheptamers by adding rabbit red cell membranes and incubating for 2 h. The heptamers were purified by SDS-polyacrylamide gel electrophoresis and stored in aliquots at −80° C.

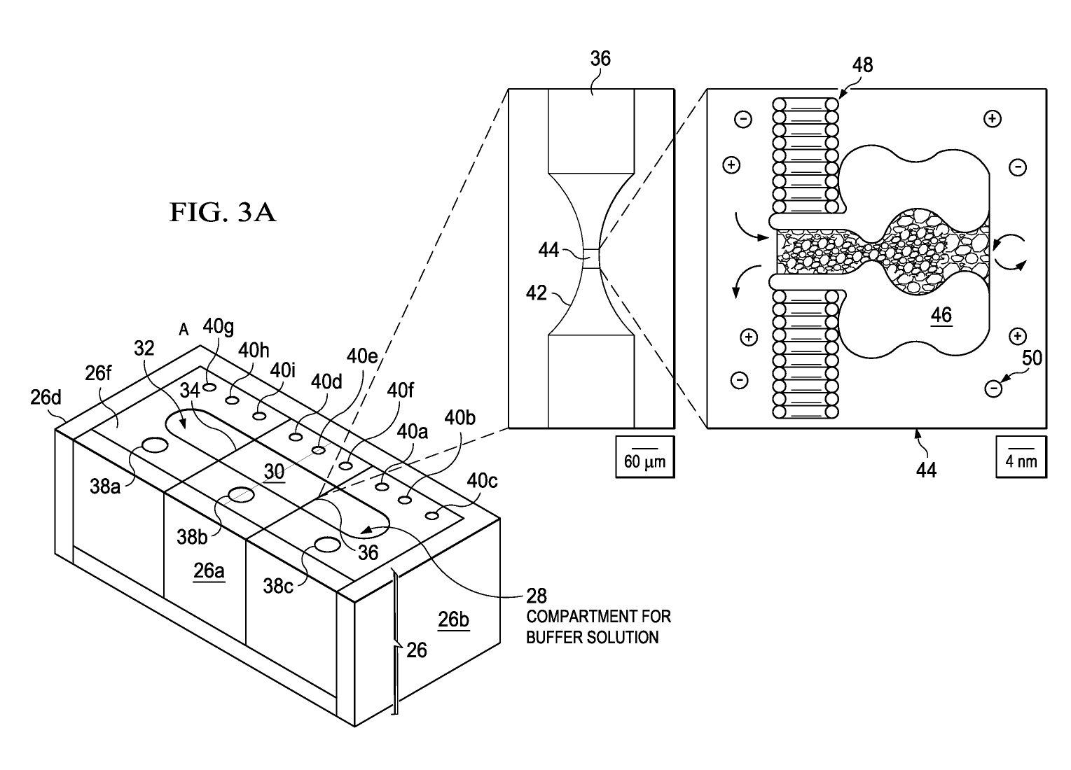

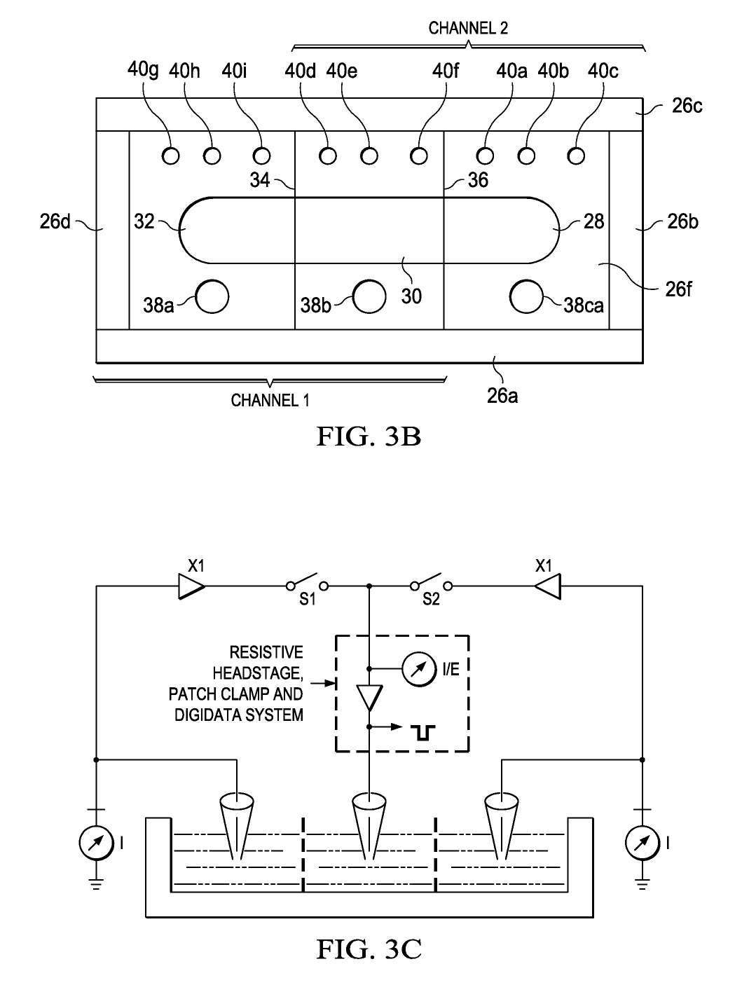

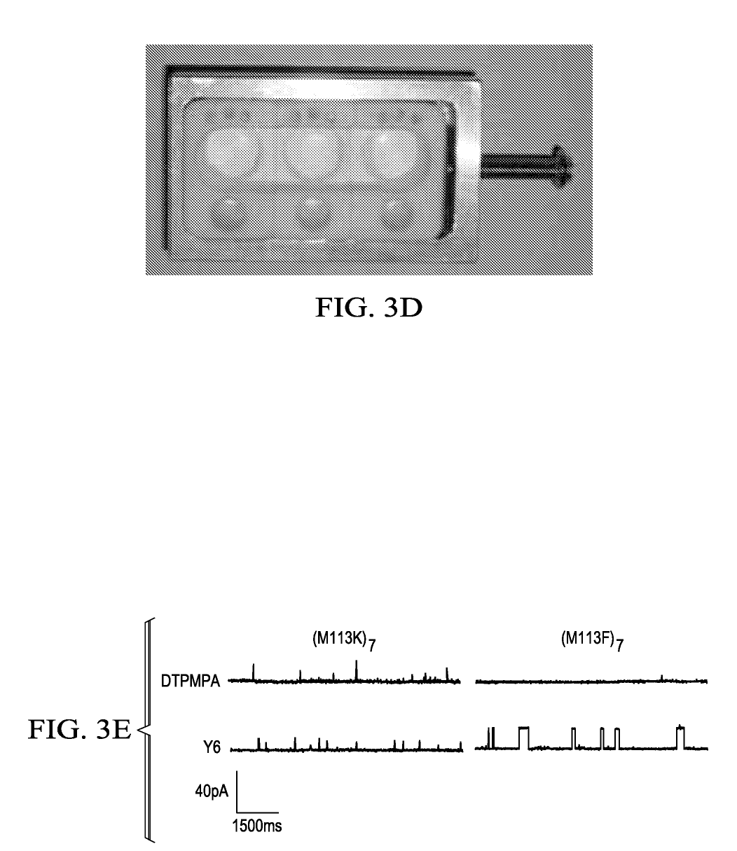

A two-nanopore sensor chamber 26 design is shown in FIG. 3A. The two-nanopore sensor chamber comprises three compartments, 28, 30 and 32 separated by two Teflon films 34 and 36. The chamber 26 is a six sided rectangular cube (26a-26f). Sides 26c and 26e are not shown. The sample is added to the middle compartment 30. There are three electrodes in the device held in holes 38a, 38b, and 38c on the top surface (26f) of the device 26. There are nine holes for adding and transferring the solutions 40a-40i.

An expanded view of the Teflon film 36 is shown and it comprises a bi-layer 42 and a single transmembrane protein α-hemolysin (αHL) channel 44. The expanded view of the transmembrane protein αHL channel 44 is also shown and it comprises a mushroom cap structure 46, and a bi-layer 48. The electrolyte to be used 50 is also shown.

FIG. 3B shows the top view of the two-pore nanopore sensor 26 of FIG. 3A. The top view shows the three compartments, 28, 30 and 32 separated by two Teflon films 34 and 36. FIG. 3B also shows the holes 38a, 38b, and 38c for the three electrodes and six holes for adding and transferring the solutions 40a-40i. Sides 26a, 26b, 26c, 26d, and 26f are also shown in FIG. 3B. Compartments 32 and 30 include channel 1 and compartments 30 and 28 comprise channel 2 of the two-nanopore sensor chamber 26.

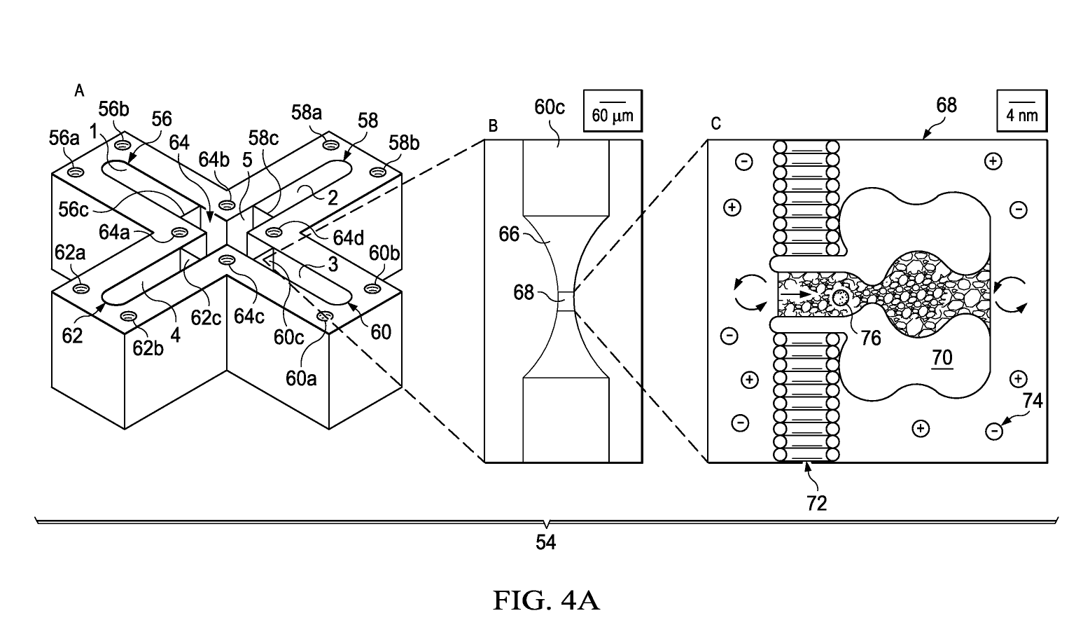

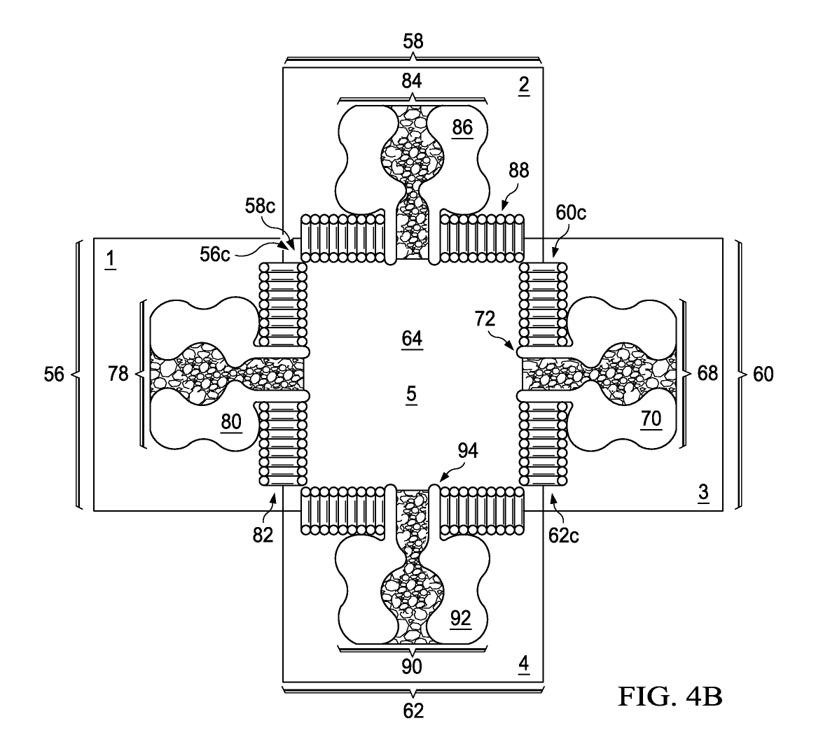

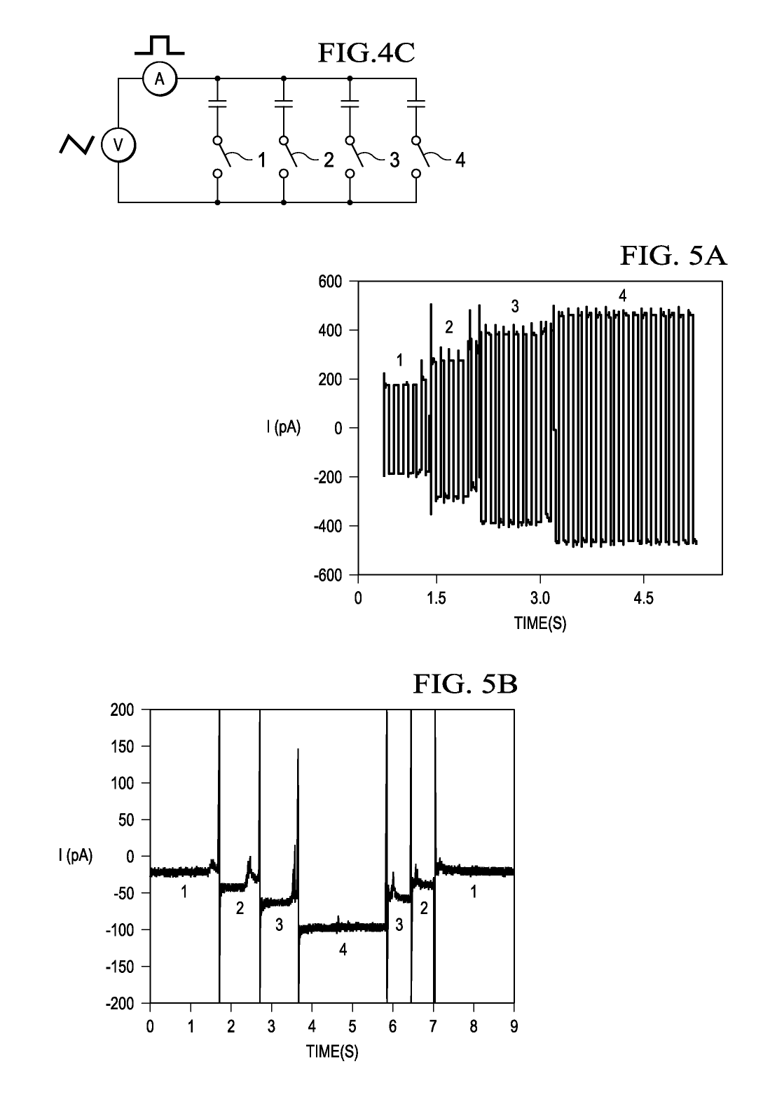

The four-nanopore sensor chamber 54 comprises of five compartments, 56, 58, 60, 62, and 64, which are separated by four Teflon films 56c, 58c, 60c, and 62c (25 μm thick; Goodfellow, Malvern, Pa., USA) with a 150 μm aperture (FIG. 4A). Four different protein pores were added to the four surrounding compartments, 56, 58, 60, and 62 while the center compartment 64 will be used to hold the sample solution and be shared by all the four nanopore sensors. In this design, the center compartment and each of the four surrounding compartments construct one individual nanopore sensor (FIG. 4B). Furthermore, five electrodes are located in this device, in holes 56a, 58a, 60a, 62a, and 64a where the central electrode 64a is shared by the four nanopore sensors (not shown), while the other four electrodes 56a-62a are grounded. There are 7 holes for adding and transferring the solutions 56b, 58b, 60b, 62b, 64b, 64c, and 64d.

In addition, a parallel circuit is employed in this pattern-recognition nanopore sensing system, where four switches are used to control which pore(s) will be monitored (FIG. 4C).

An expanded view of the Teflon film 60c is shown and it comprises a bi-layer 66 and a single transmembrane protein α-hemolysin (αHL) channel 68. The expanded view of the transmembrane protein αHL channel 68 is also shown and it comprises a mushroom cap structure 70, a bi-layer 72, and the electrolyte to be used 74. The analyte to be detected 76 is also shown.

A top view of four-nanopore sensor chamber 54 comprising the five compartments, is shown in FIG. 4B. The four compartments with the protein pores are 56, 58, 60, and 62. The sample holding compartment is 64. Each of the compartments are separated by Teflon films 56c, 58c, 60c, and 62c each comprising bi-layers 82, 88, 72, and 94 respectively, and an embedded single transmembrane protein α-hemolysin (αHL) channel 78, 84, 68, and 90, respectively. The αHL comprises a mushroom cap structure, and a β-barrel. The mushroom cap structures for the four (αHL) channels 78, 84, 68, 90 are represented by 80, 86, 70, and 92, respectively.

Single-channel current recordings were carried out as described at a temperature of 22°±1° C. [26]. Briefly, the four apertures in the four films were pretreated with 10% (v/v) hexadecane (Aldrich; Milwaukee, Wis.) in n-pentane (Burdick & Jackson; Muskegon, Mich.). Four bilayers of 10 mg/mL 1,2-diphytanoylphosphatidylcholine (Avanti Polar Lipids; Alabaster, Ala., USA) in n-pentane were formed on the apertures. The formation of the four bilayers was achieved by using the Montal-Mueller (i.e., monolayer folding) method [27], and monitored by using a function generator (BK precision 4012A; Yorba Linda, Calif., USA). To form these four bilayers, the buffer solution level of the center compartment was raised, followed by raising the fluid levels of other four surrounding compartments. The studies were performed under symmetrical buffer conditions with each compartment containing a 2.0 mL solution of 1 M NaCl and 10 mM Tris HCl (pH 7.5). Unless otherwise noted, the αHL proteins were added to the surrounding (i.e., cis) compartments, which were connected to “ground”, while peptides and/or organophosphates were added to the center (i.e., trans) compartment. In such a way, after insertion of a single αHL channel, its mushroom cap would be located in the cis compartment, while the β-barrel of the αHL would insert into the lipid bilayer and connect with the trans of the pattern-recognition nanopore chamber device. To facilitate the insertion of four concurrent channels, the insertion rates of the four protein pores were monitored, followed by addition of the corresponding concentration of each protein to one of the four chamber compartments to ensure that the waiting times for the four channel insertions did not differ significantly. The final concentrations of the αHL proteins were 0.2-2.0 ng·mL⁻¹. The transmembrane potential, which was applied with Ag/AgCl electrodes with 3% agarose bridges (Sigma) containing 3 M KCl (EMD Chemicals Inc; Darmstadt, Germany), was −40 mV. A negative potential indicates a lower potential in the trans chamber of the apparatus. Currents were recorded with a patch clamp amplifier (Axopatch 200B, Molecular Devices; Sunnyvale, Calif., USA). The currents were low-pass filtered with a built-in four-pole Bessel filter at 2 kHz and sampled at 10 kHz by a computer equipped with a Digidata 1440 A/D converter (Molecular Devices). To shield against ambient electrical noise, a metal box was used to serve as a Faraday cage, inside which the bilayer recording amplifying headstage, stirring system, chamber, and chamber holder were enclosed.

Data were analyzed with the following software: pClamp 10.0 (Molecular Devices) and Origin 7.0 (Microcal, Northampton, Mass.). Conductance values were obtained from the amplitude histograms after the peaks were fit to Gaussian functions. Mean residence times (τ values) for the analytes were obtained from dwell time histograms by fitting the distributions to single exponential functions by the Levenberg-Marquardt procedure.

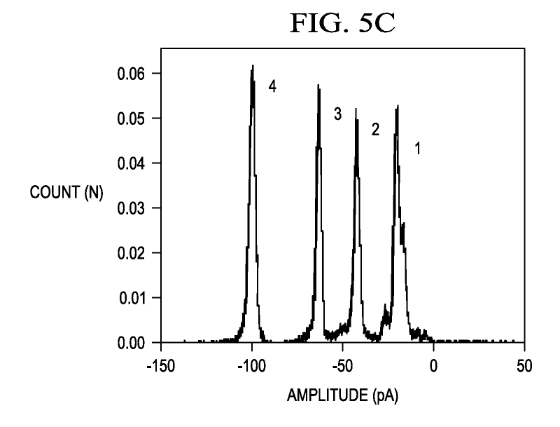

In the four-nanopore sensor configuration, the center compartment (i.e., the common sample reservoir) and each of the four surrounding “cis” compartments will comprise one individual nanopore sensor. An advantage of this nanopore sensing design is that the amount of the sample required for analysis is much smaller than the individual pore or the independent array pore approach [28]. This is an important consideration in the detection of precious biomolecule samples, e.g., DNA, peptides, and proteins. Furthermore, since our nanopore sensing system employs a parallel electric circuit of on/off switches to control which channel(s) to be monitored (FIG. 4C), each component sensor element can work independently or act together with other pores. The constructed four-nanopore sensor pattern-recognition device was employed to examine its feasibility to form four stable lipid bilayers and four concurrent single channels. To monitor whether bilayers were formed on the apertures in the four Teflon films, initially, only switch #1 was turned on. Then, the other three switches were turned on sequentially. The electric recording of the entire process, i.e., monitoring from one bilayer to four bilayers, is shown in FIG. 5A. It could be seen that once all the four bilayers were formed, the overall capacitive current was around 462 pA, which corresponds to a capacitance of approximately 560 pF according to C=I (dt/dV), where I is the current value, dt is the half period of bilayer, and dV is the applied voltage. The values obtained for dt and dV under the described experimental conditions were 48.5 ms and 40 mV, respectively.

The bilayer capacitive currents play a critical role in the single channel recording studies. Larger the current, the faster is the single channel insertion. However, a larger capacitive current value indicates that the bilayer formed on the aperture has a larger surface area and becomes less stable (note that I∞C=∈ᵣA/d, where ∈ᵣ, d, and A are the dielectric constant, thickness, and area, respectively, of the bilayer) [29]. Each bilayer current obtained was kept in the range of 100˜200 pA in the studies, which enabled both the efficient insertion of alpha-hemolysin (αHL) pores and long lifetimes of the formed bilayer membranes. Although the stabilities of the four bilayers are different, in most cases, the lifetime of each bilayer is at least three hours even after insertion of an αHL pore (note that single-channel recording studies are accomplished within minutes). FIG. 5B shows the single-channel current recordings after four different αHL pores were added to the four surrounding cis compartments. Since the switches were turned on sequentially and then turned off one by one in the experiment, this confirmed that the four channels were from four different αHL protein pores, rather than multiple channels from a single αHL protein.

To evaluate the performance of nanopore pattern-recognition, four different αHL protein pores were employed, including (M113F)₇(T145F)₇(K147N)₇, (M113E)₇, (M113R)₇(T145R)₇, and wild type (WT)₇ αHL protein pores. They were added to the four cis compartments of the sensing chamber to serve as the sensing elements (sensors 1, 2, 3, and 4, respectively). Of the protein pores used, the three mutants were genetically engineered at and/or near the position 113 of the αHL polypeptide. The position 113 is close to the narrowest part of the lumen, and has been used to design nanopore sensors for a variety of compounds [2,4-5]. The binding sites in the protein pores belonged to four major classes. The mutant (M113E)₇ pore presents an electrostatic interaction site (containing seven negatively charged Glu amino acid residues) for positively charged compounds. The engineered (M113R)₇(T145R)₇ protein contains fourteen positively charged Arg side chains, providing an interaction site for negatively charged molecules. The (M113F)₇(T145F)₇(K147N)₇ channel contains an aromatic binding site (consisting of fourteen aromatic Phe side chains) for aromatic analytes. The (WT)₇ αHL pore has seven Met residues at position 113, proving a hydrophobic interaction surface. In general, hydrophobic interactions can also occur in the three mutant αHL proteins, although the designed specificity for aromatic or charged compounds should provide a high degree of selectivity among the different variants.

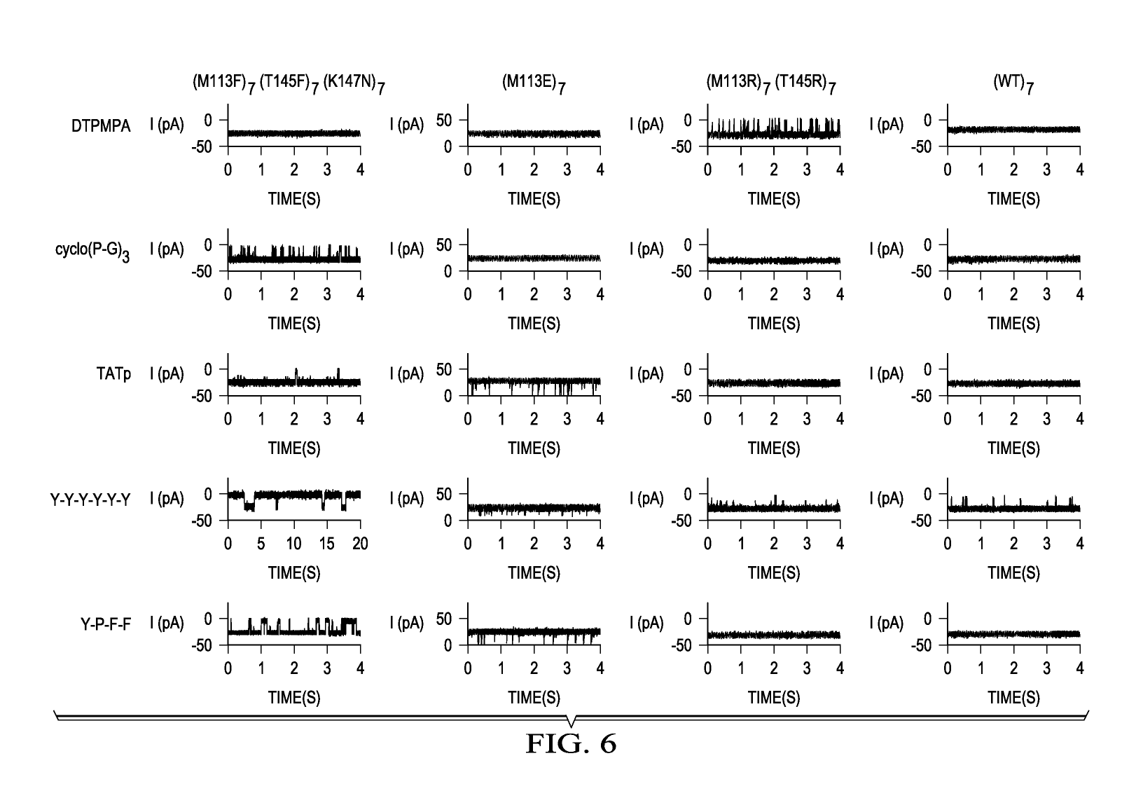

After insertion of the four protein channels, five compounds were examined. These compounds included organophosphate Diethylenetriaminepentamethylenephosphonic acid heptasodium salt (DTPMPA), as well as four peptides: cyclo(P-G)₃, Y-Y-Y-Y-Y-Y (Y6), (SEQ ID NO.: 1) Y-P-F-F (SEQ ID NO.: 2), and HIV-1 TAT protein peptide (TATp) with a sequence of Y-G-R-K-K-R-R-Q-R-R-R (SEQ ID NO.: 3). Like the protein pores used, these analytes also belonged to four major categories: hydrophobic (cyclo(Pro-Gly)₃), negatively charged (DTPMPA), positively charged (TATp), and aromatic (Y6 and Y-P-F-F (SEQ ID NO.: 2)). Single-channel recordings are shown in FIG. 6. Note again that, in our pattern-recognition nanopore studies, the response of a component nanopore to a molecule is monitored via the parallel circuit of on/off switches (FIG. 4C). Each single-channel recording was obtained with only one switch turned on in turn.

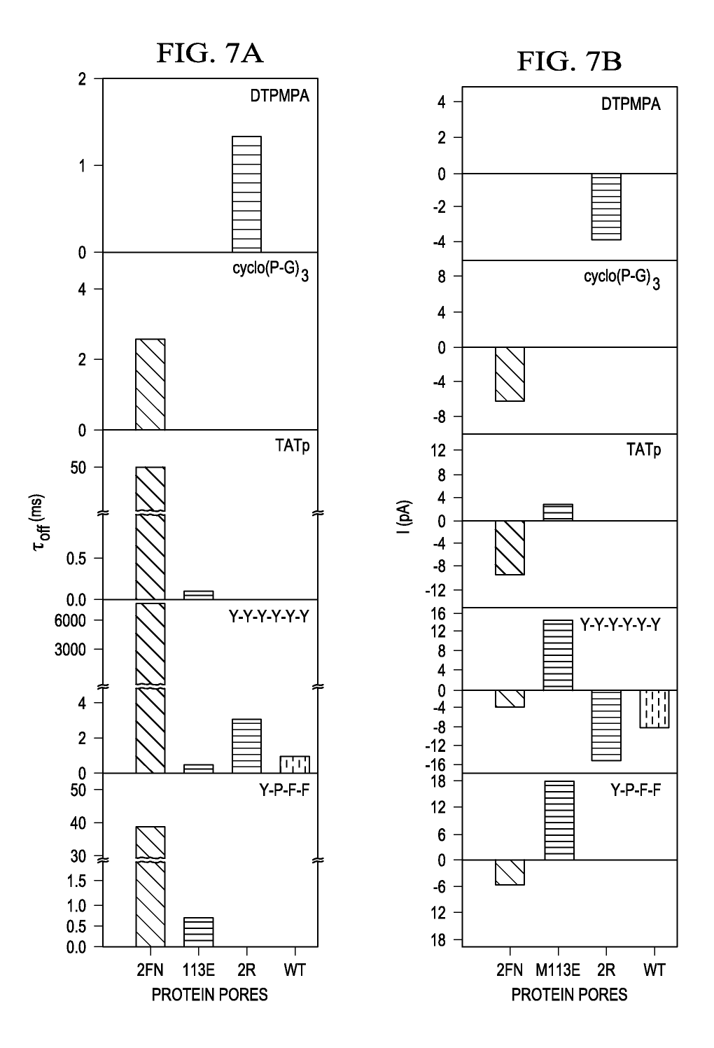

If the currently available single pore sensor approach was used, in the case of detection of peptide cyclo(P-G)₃ and organophosphate DTPMPA, only one analyte could be accurately detected. For example, if sensor 1 (i.e., the (M113F)₇(T145F)₇(K147N)₇ pore) was used, only cyclo(P-G)₃ could be identified. On the other hand, if sensor 3 (i.e., the (M113R)₇(T145R)₇ pore) was employed, only DTPMPA could be identified. Similarly, in the case of cyclo(P-G)₃ and TATp, sensor 2 (i.e., the (M113E)₇ pore) could be used to detect only TATp, while sensor 1 (i.e., the (M113F)₇(T145F)₇(K147N)₇ pore) could only accurately identify cyclo(P-G)₃, although TATp also showed a very weak response. In a mixture of cyclo(P-G)₃ and TATp, the signal of TATp will be hidden by that of cyclo(P-G)₃ when they are detected by sensor 1. In the case of TATp and Y-P-F-F (SEQ ID NO.: 2), since they produced very similar event signatures in sensor 2 (i.e., the (M113E)₇ pore), once again these two peptides could not be differentiated using a single nanopore sensor. However, by using the pattern-recognition nanopore sensor array, we can rely on the collective responses of all the component pores to a compound to produce a response pattern to differentiate all the analytes in these three cases. For example, cyclo(P-G)₃ had no signal in the sensor 3, but caused current blocking events in sensor 1. In contrast, DTPMPA had signal in sensor 3, but no signal in sensor 1. This allows the construction of a pattern-recognition plot to differentiate these two compounds. The pattern-recognition plots for all five compounds are shown in FIG. 7. Note that, either the event dwell time or amplitude or both could be employed as parameters in the plot. Clearly, all the five compounds could be differentiated by using this pattern-recognition approach.

The performance of the pattern-recognition stochastic sensor relies on the selectivity and resolution of each component nanopore. In our design, nanopore recognition is based on weak non-covalent bonding interactions, specifically hydrophobic, aromatic, and electrostatic forces. To differentiate a variety of different types of compounds, e.g., hydrophobic, aromatic, positively charged, and negatively charged, each individual pore in the pattern-recognition sensing device has different functional groups, ranging from super hydrophobic to ultra hydrophilic, as well as having both positive and negative charged surfaces. Thus, each component pore of the sensor device is different and reacts differently toward a molecule. For example, as shown in FIG. 6, the dwell time for Y6 in (WT)₇ αHL is 1.2 ms, which is larger than that of Y6 in (M113E)₇ (0.43 ms), but smaller than those in the (M113R)₇(T145R)₇ (3.1 ms) and the (M113F)₇(T145F)₇(K147N)₇ (7333.6 ms) protein pores. Generally, the strengths of the hydrophobic interactions occurring in all these nanopores depend on the van der Waals volumes of the side-chains at the binding site [30]. For example, the van der Waals volume (124 Å³) of the Met-113 residue of (WT)₇ αHL is larger than that of Glu-113 of the modified (M113E)₇ pore (91 Å³), but smaller than those of Arg-113 of the engineered (M113R)₇(T145R)₇ protein (148 Å³) and Phe-113 of the modified (M113F)₇(T145F)₇(K147N)₇ pore (135 Å³). The dwell times obtained for Y6 in the four protein pores were in agreement with the van der Waals volumes of the four amino acids except Phe, where the predominant binding affinity of Y6 to the pore is an aromatic interaction. This results in a much larger residence time of up to several seconds. It should be noted that, in the case of differentiation of compounds with similar structures and/or functions, e.g., in the analysis of a group of charged molecules, an array of nanopores with different surface charge densities should be employed as the sensing elements of the pattern-recognition sensor for better resolution. In addition, for the detection of positively and negatively charged molecules using the pattern-recognition design, opposite voltage is applied since these charged molecules need to be electrophoretically driven into the pore and then interact with the binding site. This causes a concern regarding the utility of the sensor to sense positively and negatively charged molecules in a single experiment. However, this issue can be readily resolved by measuring the sample with both positive and negative applied potentials. The polarity can be conveniently changed with the patch clamp amplifier.

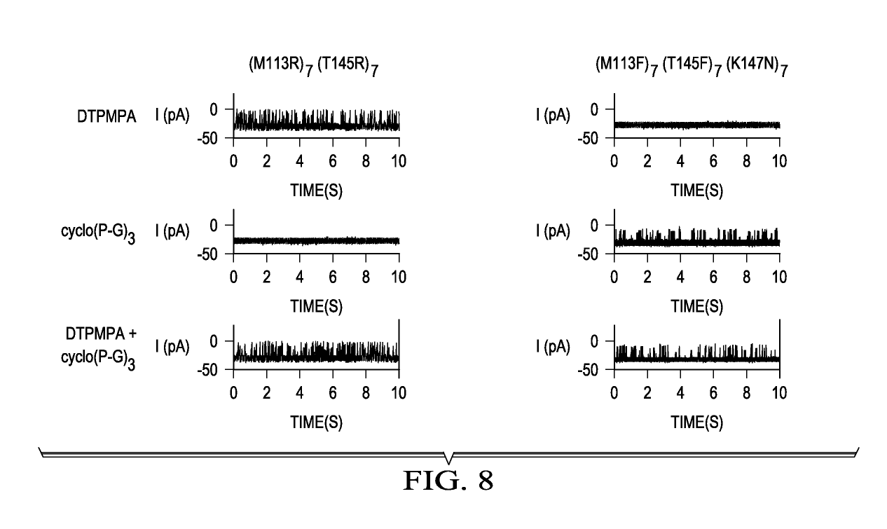

Identification and quantitative determination of biomolecules, typically present at very low concentrations in complex mixtures, is a topic of intense. Furthermore, the availability of a high-throughput method for multi-analyte detection would have enormous implications in terms of sensor technology including environmental monitoring, drug discovery, medical diagnosis, and homeland security [31, 32]. For this purpose, a pattern-recognition nanopore sensor consisting of two component pores was used to detect two compounds. The experimental results (FIG. 8) showed that, if the sample contained only one component (either DTPMPA or cyclo(P-G)₃), its current blocking events were observed with only one of the corresponding sensors. In contrast, if a mixture of DTPMPA and cyclo(P-G)₃ was present in the sample, the blocking events were identified in both the sensor pores, thus allowing the simultaneous detection of the mixture. Since only one component of the mixture could be identified using a single pore, it provides further evidence that the nanopore array enabled the enhanced resolution to detect the mixture than the currently available single pore configuration. In this particular study, the two nanopores only responded to one analyte each. However, in the case of an array of nanopores with specific recognition elements employed as the component sensing elements, the constructed sensor array can readily detect a mixture of compounds.

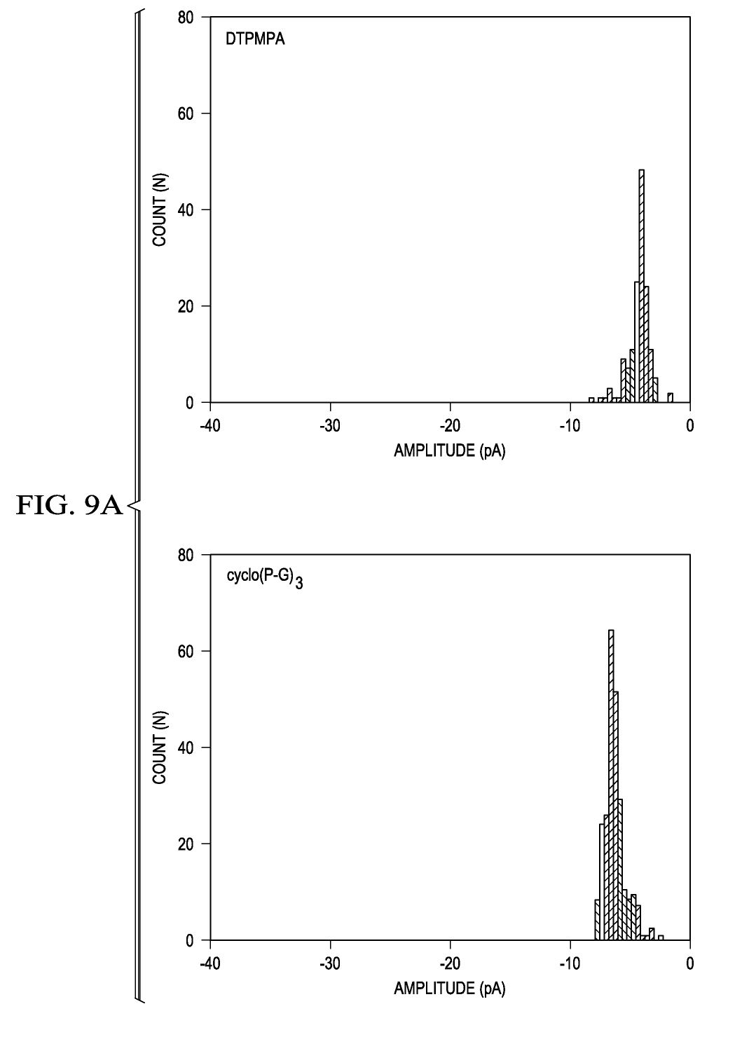

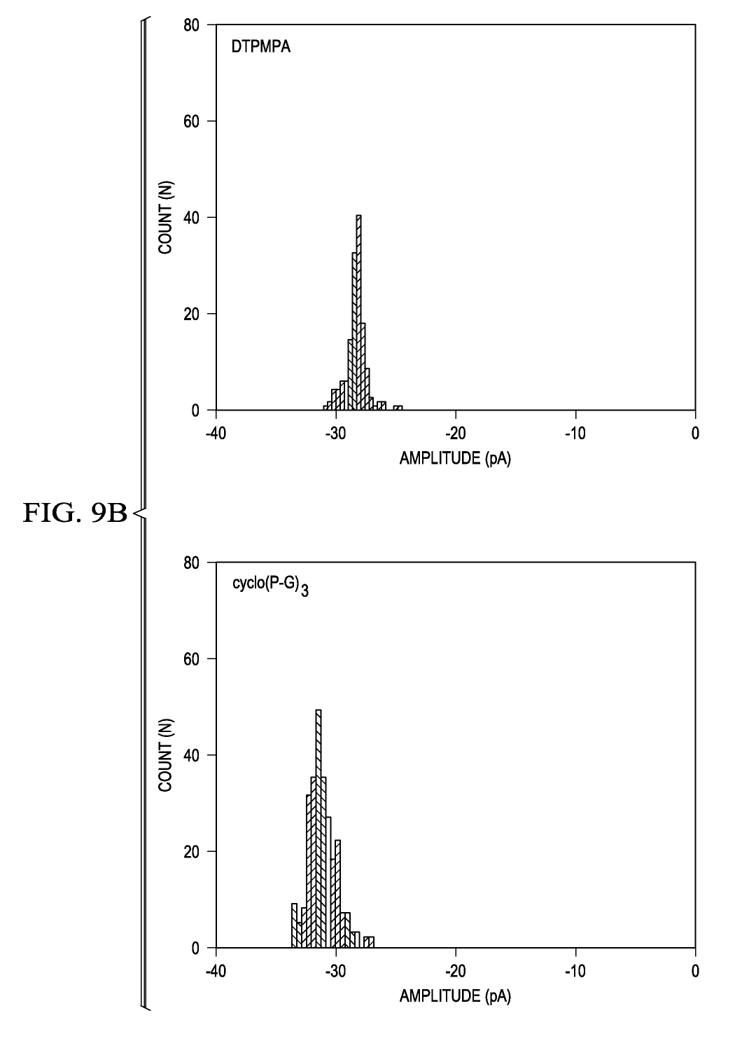

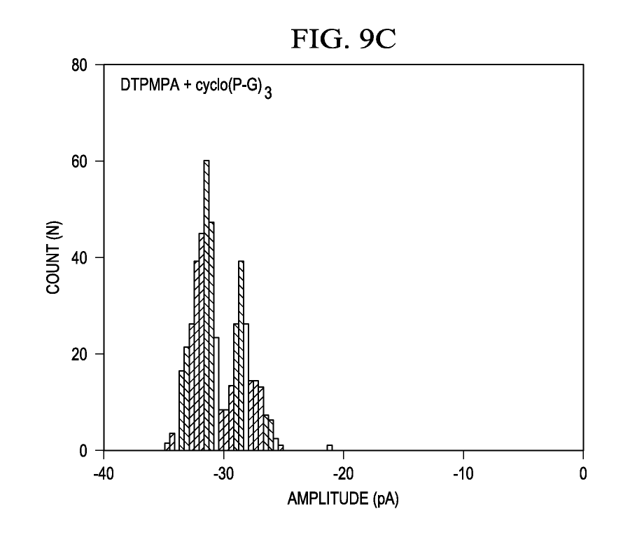

One of the advantages of the array nanopore design in the present invention is that it could be conveniently converted into a multiple-pore/multiple-analyte sensor if the switches of the parallel circuit are turned on concurrently, allowing simultaneous detection of a mixture of compounds in a single trace. As shown in FIGS. 8 and 9a, the open channel currents of sensor 1 and sensor 2 were −27.1 pA, and −28.3 pA, respectively, while the mean residual currents of DTPMPA events in sensor 1 and cyclo(P-G)₃ events in sensor 2 were −4.0 pA, and −6.4 pA, respectively. In a double-channel consisting of such two different single pores (i.e., sensors 1 and 2), the combined open channel current was increased to −51.0 pA. In the presence of a single DTPMPA or cyclo(P-G)₃ component, they caused current blockage events with a mean residual current of −28.1 pA, and −31.3 pA, respectively (FIG. 9b). In contrast, two types of events with residual currents of −28.6 pA and −31.6 pA were observed when a sample containing a mixture of DTPMPA and cyclo(P-G)₃ was added to the common trans compartment (FIG. 9c).

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

U.S. Pat. No. 7,005,264: Method and apparatus for nucleic acid sequencing and identification.
United States Patent Application No. 20070178507: Method and apparatus for detection of molecules using nanopores.
1. Song, L.; Hobaugh, M. R.; Shustak, C.; Cheley, S.; Bayley, H.; Gouaux, J. E. 1996 Structure of staphylococcal alpha-hemolysin, a heptameric transmembrane pore Science 274 1859-1866.
2. Cheley, S.; Gu, L.-Q.; Bayley, H. 2002 Stochastic sensing of nanomolar inositol Chem. Biol. 9 829-838.
3. Gu, L.-Q.; Braha, O.; Conlan, S.; Cheley, S.; Bayley, H. 1999 Stochastic sensing of organic analytes by a pore-forming protein containing a molecular adapter Nature 398 686-690.
4. Guan, X.; Gu, L.-Q.; Cheley, S.; Braha, O.; Bayley, H. 2005 Stochastic sensing of TNT with a genetically engineered pore ChemBioChem 6 1875-1881.
5. Kang, X. F.; Cheley, S.; Guan, X.; Bayley, H. 2006 Stochastic detection of enantiomers J. Am. Chem. Soc. 128 10684-10685.
6. Movileanu, L.; Howorka, S.; Braha, O.; Bayley, H. 2000 Detecting protein analytes that modulate transmembrane movement of a polymer chain within a single protein pore Nat. Biotechnol. 18 1091-1095.
7. Howorka, S.; Cheley, S.; Bayley, H. 2001 Sequence-specific detection of individual DNA strands using engineered nanopores Nat. Biotechnol. 19 636-639.
8. Shin, S.-H.; Luchian, T.; Cheley, S.; Braha, O.; Bayley, H. 2002 Kinetics of a reversible covalent-bond-forming reaction observed at the single-molecule level Angew. Chem. 114 3859-3861.
9. Braha, O.; Gu, L.-Q.; Zhou, L.; Lu, X.; Cheley, S.; Bayley, H. 2000 Simultaneous stochastic sensing of divalent metal ions Nat. Biotechnol. 17 1005-1007.
10. Dickinson, T. A.; White, J.; Kauer, J. S.; Walt, D. R. 1998 Current trends in ‘artificial-nose’ technology Trends Biotechnol. 16 250-258.
11. Turner, A. P.; Magan, N. 2004 Electronic noses and disease diagnostics Nat. Rev. Microbiol. 2 161-166.
12. Thaler, E. R.; Kennedy, D. W.; Hanson, C. W. 2001 Medical applications of electronic nose technology: review of current status Am. J. Rhinol. 15 291-295.
13. Bayley, H.; Cremer, P. S. 2001 Stochastic sensors inspired by biology Nature 413 226-230.
14. Carey, W. P.; Beebe, K. R.; Kowalski, B. R. 1987 Multicomponent analysis using an array of piezoelectric crystal sensors Anal. Chem. 59 1529-1534.
15. Icco, A. J.; Crooks, R. M.; Osbourn, G. C. 1998 Surface acoustic wave chemical sensor arrays: new chemically sensitive interfaces combined with novel cluster analysis to detect volatile organic compounds and mixtures Acc. Chem. Res. 31 289-296.
16. Chang, S-C.; Pereira-Rodrigues, N; Henderson, J. R.; Cole, A; Bedioui, F; McNeil, C. J. 2005 An electrochemical sensor array system for the direct, simultaneous in vitro monitoring of nitric oxide and superoxide production by cultured cells Biosens. Bioelectron. 21 917-922.
17. Ferreira, M; Riul, A Jr.; Wohnrath, K.; Fonseca, F. J.; Oliveira, O. N. Jr.; Mattoso, L. H. 2003 High-performance taste sensor made from Langmuir-Blodgett films of conducting polymers and a ruthenium complex Anal. Chem. 75 953-955.
18. Greene, N. T.; Shimizu, K. D. 2005 Colorimetric molecularly imprinted polymer sensor array using dye displacement J. Am. Chem. Soc. 127 5695-5700.
19. Li, J.; Stein, D.; McMullan, C.; Branton, D.; Aziz, M. J.; Golovchenko, J. A. 2001 Ion-beam sculpting at nanometre length scales Nature 412 166-169.
20. Sandhu, A. 2007 New probes offer much faster results Nature Nanotechnol 2 746-748.
21. Zhao, Q.; Sigalov, G.; Dimitrov, V.; Dorvel, B.; Mirsaidov, U.; Sligar, S.; Aksimentiev, A.; Timp, G. 2007 Detecting SNPs using a synthetic nanopore Nano Lett. 7 1680-1685.
22. Benner S, Chen R J A, Wilson N A, Abu-Shumays R, Hurt N, Lieberman K, Deamer D W, Dunbar W B and Akeson M 2007 Sequence-specific detection of individual DNA polymerase complexes in real time using a nanopore Nature Nanotech. 2 718-24
23. Meller, A.; Nivon, L.; Brandin, E.; Golovchenko, J.; Branton, D. 2000 Rapid nanopore discrimination between single polynucleotide molecules Proc. Natl. Acad. Sci. USA 97 1079-1084.
24. Sanchez-Quesada, J.; Saghatelian, A.; Cheley, S.;, Bayley, H.; Ghadiri, M. R. 2004 Single DNA rotaxanes of a transmembrane pore protein Angew. Chem. Int. Ed. Engl. 43 3063-3067.
25. Kasianowicz, J. J.; Brandin, E.; Branton, D.; Deamer, D. 1996 Characterization of individual olynucleotide molecules using a membrane channel Proc. Natl. Acad. Sci. USA 93 13770-13773.
26. Zhao Q, Jayawardhana D A and Guan X 2008 Stochastic study of the effect of ionic strength on noncovalent interactions in protein pores Biophys. J. 94 1267-1275.
27. Montal M and Mueller P 1972 Formation of bimolecular membranes from lipid monolayers and a study of their electrical properties Proc. Natl. Acad. Sci. U.S.A. 69 3561-3566.
28. Kang, M.; Trofin, L.; Mota, M. O.; Martin, C. R. 2005 Anal. Chem. 77 6243-6249.
29. White, R. J.; Ervin, E. N.; Yang, T. L.; Chen, X.; Daniel, S.; Cremer, P. S.; White, H. S. 2007 Single ion-channel recordings using glass nanopore membranes J. Am. Chem. Soc. 129 11766-11775.
30. Creighton, T. E. 1993 Proteins: structures and molecular properties (New York: W.H. Freeman).
31. Palacios, M. A.; Nishiyabu, R.; Marquez, M.; Anzenbacher, P. Jr. 2007 Supramolecular Chemistry Approach to the Design of a High-Resolution Sensor Array for Multianion Detection in Water. J. Am. Chem. Soc. 129, 7538-7544.
32. Folmer-Andersen, J. F.; Kitamura, M.; Anslyn, E. V. 2006 Pattern-based discrimination of enantiomeric and structurally similar amino acids: an optical mimic of the mammalian taste response. J. Am. Chem. Soc. 128, 5652-5653.

What is claimed is:

1. A single molecule chemical sensing apparatus comprising:
at least two cis chambers;
at least one trans chamber;
at least two boundary layers on a septum separating the cis and trans chambers;
at least one pore selected from a porous synthetic membrane, or a wild type or genetically modified bacterial transmembrane protein attached to the boundary layer;
one or more holes for addition of one or more solutions to the one or more chambers;
one or more holes for placing one or more electrodes to the one or more chambers;
at least three electrodes for establishing an electric potential;
at least two or more switches for monitoring an ionic current output; and
an enclosure for the single molecule chemical sensing apparatus.

2. The apparatus of claim 1, wherein the septum has a hole having a diameter ranging from 100-200 μm.

3. The apparatus of claim 1, wherein a conducting electrolyte is present in at least one of the chambers.

4. The apparatus of claim 1, wherein an analyte is present in at least one of the chambers.

5. The apparatus of claim 1, wherein the boundary layer comprises a lipid bi-layer or a natural or synthetic membrane.

6. The apparatus of claim 1, wherein the ionic current output is measured from at least two chambers sequentially or simultaneously.

7. The apparatus of claim 1, wherein the wild type or modified bacterial transmembrane protein comprises at least one or more of α-hemolysin, streptolysin, listeriolysin, leukocidin, binary toxins, aerolysin, cholesterol-dependent cytolysins, pneumolysins, or combinations thereof.

8. The apparatus of claim 3, wherein the conducting electrolyte comprises a buffer, ionic salts, organic ion conducting solutions or combinations thereof.

9. The apparatus of claim 4, wherein the analytes are detected by a priori knowledge, statistical patterns, multidimensional spatial analysis, or combinations thereof.

10. The apparatus of claim 4, wherein the analytes unknown, known, or combinations thereof.

11. The apparatus of claim 4, wherein the analyte solutions comprises biomolecules, oligonucleotides, environmental contaminants, bioterrorist agents, or combinations thereof.

12. The apparatus of claim 4, wherein the analyte is a biomolecule, comprising one or more proteins, peptides, fusion proteins, cells, monoclonal antibodies, polyclonal antibodies, receptors, growth-factors, hormones, or combinations thereof.

13. The apparatus of claim 4, wherein the analyte is a bioterrorist agent, comprising one or more toxins, liquid explosives, toxins including neurotoxins and anthrax, cholinergic agents, TNT, or combinations thereof.

14. The apparatus of claim 4, wherein the analyte is an environmental contaminant, comprising one or more, heavy metals, cations, toxic chemicals, polymeric compounds, or combinations thereof.

15. The apparatus of claim 4, wherein the analyte is an oligonucleotide, comprising one or more, ssDNA, RNA, double stranded DNA, polynucleotides, or combinations thereof.

16. The apparatus of claim 1, wherein the one or more genetically modified bacterial transmembrane protein toxin and made by cassette mutagenesis comprising the steps of:
cleaving a bacterial plasmid by a restriction enzyme to form an excised internal fragment and a plasmid with sticky ends;
replacing the excised internal fragment by an oligonucleotide containing a sense and an antisense fragment; and
inserting by ligation the sticky ends of the bacterial plasmid and the oligonucleotide to form a genetically modified bacterial transmembrane protein toxin.

17. The apparatus of claim 16, wherein the restriction enzyme comprises, one or more enzymes selected from EcoRI, EcoRII, BamHI, HindIII, TaqI, NotI, HinfI, Sau3A, PovII, SmaI, HaeIII, AluI, HpaI, SacII, EcoRV, KpnI, PsfI, SacI, SalI, ScaI, SphI, StuI, XbaI, and combinations thereof.

18. The apparatus of claim 1, wherein the one or more genetically modified bacterial transmembrane α-hemolysin are produced by cassette mutagenesis comprising the steps of:
cleaving a bacterial plasmid pT7-αHL-RL2 position by restriction enzymes SacII and HpaI to form an excised fragment and a plasmid with sticky ends;
replacing the excised internal fragment with a duplex DNA formed comprising a sense and antisense fragments; and
inserting by ligation the sticky ends of the bacterial plasmid and the duplex DNA to form a genetically modified transmembrane α-hemolysin.

19. A method of detecting the presence of one or more single-molecules utilizing a multi-channel chemical sensing apparatus comprising the steps of:
dissolving the one or more analytes in the sample in water or a buffer solution comprising an ionic salt to form a solution;
placing the solution in a trans compartment of a multi-channel sensor;
contacting the solution with at least two or more pore assemblies comprising a wild type or genetically modified bacterial transmembrane protein toxin;
applying an electrical potential to the multi-channel sensor;
determining an ionic current across the electrical potential;
measuring one or more transient blockades in the ionic current; and
comparing the transient blockades in the ionic current to one or more known transient current blockades to determine the identity of the one or more analytes.

20. A method for fabricating a multi-channel chemical sensing apparatus for detecting single molecules, comprising the steps of:
depositing at least two bilayers of a lipid molecule in an aperture of at least two or more Teflon septa;
forming the bilayer at an air-water interface by hydrophobic apposition and the joining of the hydrocarbon chains of the individual monolayers;
monitoring the bilayer formation using a function generator;
adding at least two or more pore selected from a wild type bacterial transmembrane protein or a modified bacterial transmembrane protein to at least two or more of the bilayers or utilizing porous synthetic membranes;
adding the conducting electrolyte to the chambers;
drilling one or more holes for adding one or more solutions;
drilling one or more holes for placing at least three electrodes;
attaching at least two switches; and
enclosing the apparatus in a metal box.


SEQUENCE LISTING (3 sequences)

SEQ ID NO: 1 - protein, 6 aa, Artificial
  Synthetic protein
YYYYYY

SEQ ID NO: 2 - protein, 4 aa, Artificial
  Synthetic protein
YPFF

SEQ ID NO: 3 - protein, 11 aa, Artificial
  Synthetic Protein
YGRKKRRQRRR